(19) United States
(12) Patent Application Publication
(10) Pub. No.: US 2022/0304897 A1
(43) Pub. Date: Sep. 29, 2022

(54) TRANSABDOMINAL GASTRIC DRAINAGE DEVICE AND FACILITATOR

(71) Applicant: The Johns Hopkins University, Baltimore, MD (US)

(72) Inventor: Sandra R. DIBRITO, Houston, TX (US)

(73) Assignee: The Johns Hopkins University, Baltimore, MD (US)

(21) Appl. No.: 17/596,937

(22) PCT Filed: May 15, 2020
(86) PCT No.: PCT/US2020/033172

§ 371 Date: Dec. 22, 2021

Related U.S. Application Data
(60) Provisional application No. 62/875,165, filed on Jul. 17, 2019.

(51) Int. Cl. A61J 15/00; A61M 1/00

(52) U.S. Cl. CPC A61J 15/0015; A61J 15/0042; A61J 15/0061; A61J 15/0073; A61J 15/0096; A61M 1/87; A61M 2210/1053

(57) ABSTRACT

A gastric drainage device includes a tube with a drainage lumen and a hub to receive an inflation device. The tube includes an intragastric portion having a set of holes to permit gastrointestinal contents to pass from a stomach cavity into the drainage lumen. The gastric drainage device includes a bolster with a cylindrical port to receive the tube and a disc to hold the tube in place on a skin side of a gastrostomy site. The gastric drainage device includes a balloon formed around the intragastric portion of the tube, wherein the balloon and the intragastric portion of the tube may be passed through the gastrostomy site into the stomach cavity while the balloon is deflated. Accordingly, the balloon is inflatable within the stomach cavity using the inflation device to secure the gastric drainage device at the gastrostomy site while the gastrointestinal contents are drained from the stomach cavity.

### RELATED APPLICATION

This application claims priority to U.S. Provisional Patent Application No. 62/875,165, filed on Jul. 17, 2019, the content of which is incorporated by reference herein in its entirety.

### BACKGROUND

Gastrostomy is a procedure in which an external opening (or stoma) is created into the stomach, usually for the purpose of inserting a feeding tube through the skin and stomach wall. Gastrostomy is typically performed to provide nutrition to a patient that cannot eat or otherwise obtain nutrition orally. For example, feeding via a gastrostomy tube may be indicated for conditions such as prematurity, malnutrition, neurologic and/or neuromuscular disorders, inability to swallow, digestive disorders, cancers that obstruct the esophagus, and/or the like.

### SUMMARY

According to some implementations, a device may include: a tube including a drainage lumen and a hub adapted to receive an inflation device, wherein the tube includes an intragastric portion having a set of holes to permit gastrointestinal contents to pass from a stomach cavity into the drainage lumen; a bolster including a cylindrical port adapted to receive the tube and a disc adapted to hold the tube in place on a skin side of a gastrostomy site; and a balloon formed around the intragastric portion of the tube at a location proximal to a transabdominal portion of the tube, wherein the balloon and the intragastric portion of the tube are adapted to pass through the gastrostomy site into the stomach cavity while the balloon is deflated, and wherein the balloon is inflatable within the stomach cavity using the inflation device to secure the transabdominal portion of the tube within the gastrostomy site.

According to some implementations, a device may include: a cylindrical port adapted to receive a nasogastric tube, wherein the cylindrical port includes an extra-anatomical portion coupled to a hub adapted to receive an inflation device, and wherein the cylindrical port includes a flexible transabdominal portion; a bolster disc adapted to hold the nasogastric tube in place on a skin side of a gastrostomy site; and a balloon in communication with the hub, wherein the balloon and the flexible transabdominal portion of the cylindrical port are adapted to pass through the gastrostomy site while the balloon is deflated, and wherein the balloon is inflatable within a stomach cavity using the inflation device to secure the device and the nasogastric tube within the gastrostomy site.

According to some implementations, a system may include: a gastric drainage tube; and a facilitator device including: a cylindrical port adapted to receive the gastric drainage tube, wherein the cylindrical port includes an extra-anatomical portion coupled to a hub adapted to receive an inflation device, and wherein the cylindrical port includes a flexible transabdominal portion; a bolster disc adapted to hold the gastric drainage tube in place on a skin side of a gastrostomy site; and a balloon in communication with the hub, wherein the balloon and the flexible transabdominal portion of the cylindrical port are adapted to pass through the gastrostomy site while the balloon is deflated, and wherein the balloon is inflatable within a stomach cavity using the inflation device, after the balloon has passed through the gastrostomy site, to secure the facilitator device and the gastric drainage tube within the gastrostomy site.

### BRIEF DESCRIPTION OF THE DRAWINGS

FIG. 1 is a diagram of an example implementation of a transabdominal gastric drainage device described herein.

FIG. 2 is a diagram of an example implementation of a transabdominal gastric drainage system described herein.

### DETAILED DESCRIPTION

The following detailed description of example implementations refers to the accompanying drawings. The same reference numbers in different drawings may identify the same or similar elements.

Intestinal obstruction is a blockage in the intestines that prevents food and/or liquid from passing through the small or large intestines. In some cases, intestinal obstruction can arise when fibrous bands of tissue form in the abdomen after surgery, due to an inflamed intestine (e.g., Crohn's disease), infected pouches in the intestine, hernias, colon cancer, and/or the like. Additionally, or alternatively, an ileus can lead to an intestinal obstruction. In particular, an ileus generally refers to a condition in which a lack of movement in the intestines leads to food material building up and potentially blocking the intestines. For example, an ileus may arise when muscle or nerve problems slow down or otherwise interfere with the process of peristalsis, which normally moves digested food through the gastrointestinal tract via muscle contractions.

Accordingly, when a patient experiences an ileus and/or intestinal obstruction that interferes with food material, liquids, gases, and/or the like passing through the intestines in the normal manner, the ileus and/or intestinal obstruction can cause upstream back-up of fluids within the gastrointestinal tract, resulting in fluid pooling in the stomach. In addition to causing abdominal cramping, constipation, stomach swelling, discomfort, and/or the like for the patient, gastric distension often leads to nausea and emesis, which puts the patient at risk for aspirating gastric contents and experiencing other complications. Nasogastric tubes are often used to provide symptomatic relief and decompression of gastric distension. Nasogastric aspiration can also be used in poisoning situations when a potentially toxic liquid has been ingested, to prepare a patient before surgery under anesthesia, to extract samples of gastric liquid for analysis, and/or the like. However, nasogastric tubes, which are typically inserted in the nostril of a patient and passed into the stomach in a process known as nasogastric intubation, can pose various treatment challenges. For example, nasogastric tubes may remain in place for days, weeks, or longer, and many patients either intentionally or accidentally remove the nasogastric tube. This can result in a need to replace the nasogastric tube, in addition to potentially exposing the patient to harms associated with interrupted drainage and reinsertion (e.g., aspiration, esophageal and/or pharyngeal injury, epistaxis, and/or the like).

Some patients have gastrostomy feeding tubes, which provide a direct connection from the stomach cavity to the outside world, allowing for feeds to pass directly into the stomach, bypassing the pharynx and esophagus. Although the feeding tubes are occasionally used to “vent” the stomach in situations of obstruction, the typical design of a feeding tube hinders an ability to use the feeding tube to effectively drain gastric fluids, as feeding tubes are designed to provide feeding into the stomach rather than to remove contents from the stomach. For a patient with a gastrostomy feeding tube who is suffering from ileus or intestinal obstruction, the conventional placement of a nasogastric tube that passes through the nose and into the stomach is redundant and unnecessarily invasive when there is already a connection from the stomach to the skin. However, placing a conventional nasogastric tube through the opening (or stoma) at the gastrostomy site is suboptimal, as there is not a method to appropriately attach the nasogastric tube to the skin. Consequently, the nasogastric tube easily slips out of the stoma.

Some implementations described herein relate to a transabdominal gastric drainage device that may be used to drain gastric contents, provide gastric decompression, and/or the like for a patient having an existing gastrostomy site (e.g., an abdominal stoma). For example, in some implementations, the transabdominal gastric drainage device may include a drainage tube integrated with a bolster that has a cylindrical portion to receive the drainage tube and a disc to hold the tube in place on a skin side of the gastrostomy site. The transabdominal gastric drainage device may further include a balloon formed around an intragastric portion of the drainage tube, which can be passed through the gastrostomy site and into the stomach cavity while the balloon is deflated. Accordingly, the balloon may be inflated within the stomach cavity to secure the transabdominal gastric drainage device at the gastrostomy site, and the drainage tube may include a set of drainage holes to receive stomach contents, which are then removed from the stomach through a drainage lumen. In this way, when a patient with a gastrostomy feeding tube in place develops obstructive symptoms, the gastrostomy feeding tube may be removed and replaced with the transabdominal gastric drainage device described herein. In particular, the transabdominal gastric drainage device may be placed through the gastrostomy site, extending several centimeters into the stomach with the drainage holes arranged throughout an intraluminal length to optimize drainage. The drainage lumen may be connected to wall suction outside the body, left open to gravity, and/or the like to permit the stomach contents to be drained. The drainage tube may also include a ventilation lumen to allow air to flow into the stomach cavity as gastric contents are removed via the drainage lumen to prevent the stomach from collapsing as the stomach contents are drained via the drainage lumen.

Furthermore, some implementations described herein relate to a facilitator device that may operate as a conduit to receive and hold a gastric drainage tube (e.g., a conventional nasogastric tube) at an existing gastrostomy site (or stoma). The facilitator device may include a cylindrical port to receive the gastric drainage tube, and the cylindrical port may include an extra-anatomical portion coupled to a hub that may receive an inflation device (e.g. a Luer lock syringe). The cylindrical port may include a flexible transabdominal portion, and the facilitator device may include a balloon in communication with the hub via an inflation/deflation tube. Accordingly, the balloon and the flexible transabdominal portion may be passed through the gastrostomy site while the balloon is deflated, and the balloon may be inflated within the stomach cavity to secure the device and the gastric drainage tube at the gastrostomy site. Stomach contents can then be drained via the gastric drainage tube, and the facilitator device may include a disc that may secure the gastric drainage tube in place on a skin side of the gastrostomy site after the balloon has been inflated. After the stomach contents have been suitably drained, the balloon may be deflated, which may also cause the flexible transabdominal portion to collapse, and the facilitator device may be removed from the gastrostomy site.

In this way, the transabdominal gastric drainage device and the facilitator device described herein may avoid a need for invasive nasogastric intubation using a nasogastric tube to drain stomach contents when a patient with an existing gastrostomy site develops obstructive symptoms. In this way, the transabdominal gastric drainage device and the facilitator device described herein reduce a likelihood that the tube used for drainage will be removed, whether accidentally or intentionally. Furthermore, in this way, the transabdominal gastric drainage device and the facilitator device described herein avoid potential harms that may be associated with interrupted gastric drainage and re-insertion of the drainage tube, which could include aspiration, esophageal injury, pharyngeal injury, epistaxis, and/or the like. Further still, the transabdominal gastric drainage device and the facilitator device described herein may be more effective at draining stomach contents than using a conventional nasogastric tube at the gastrostomy site because the bolster and balloon mechanisms hold the drainage tube in place and at a correct depth within the stomach cavity.

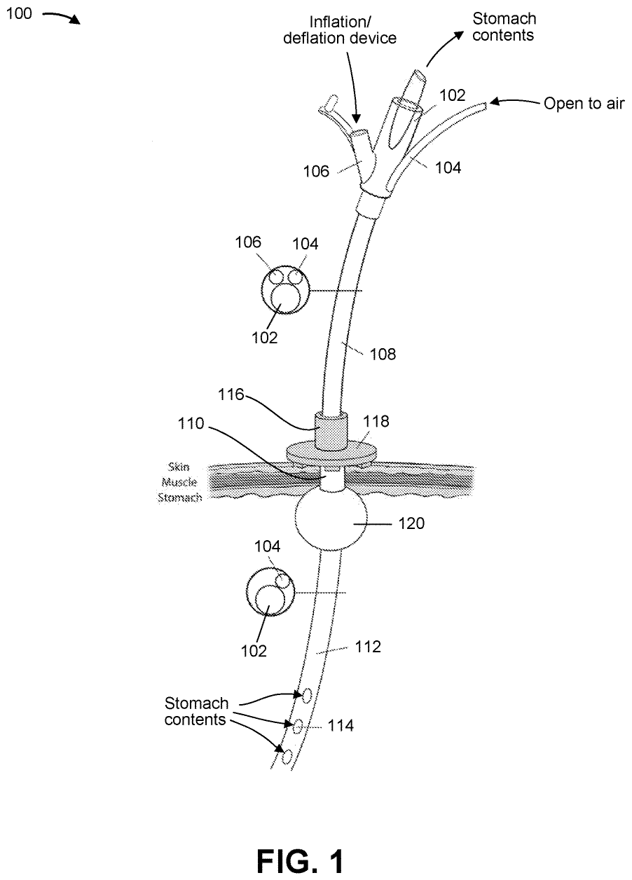

FIG. 1 is a diagram of an example implementation of a transabdominal gastric drainage device 100 described herein. As shown in FIG. 1, the transabdominal gastric drainage device 100 includes a drainage tube with a drainage lumen 102 to drain stomach contents from a patient having an existing gastrostomy site (or abdominal stoma), a ventilation lumen 104 that is open to air to permit air to pass into the stomach cavity of the patient as the stomach contents are removed via the drainage lumen 102, and a hub 106 adapted to receive an inflation and/or deflation device (e.g., a syringe filled with air, such as a Luer lock syringe). As further shown in FIG. 1, the drainage tube includes an extra-anatomical (or external) portion 108, a transabdominal portion 110, and an intragastric portion 112 that has a set of drainage holes 114 that are adapted to receive the stomach contents to be drained. For example, in FIG. 1, a cross-sectional image of the extra-anatomical portion 108 of the drainage tube shows an arrangement of the drainage lumen 102, the ventilation lumen 104, and an inflation tube coupled to the hub 106, and a cross-sectional image of the intragastric portion 112 shows continuation of the drainage lumen 102 and the ventilation lumen 104 into the stomach cavity but not the inflation tube, which ends at the transabdominal portion 110 of the drainage tube before feeding into the balloon 120. Furthermore, in some implementations, the drainage tube may include a radiopaque line to make the transabdominal gastric drainage device 100 visible in medical images (e.g., X-rays, magnetic resonance images, computed tomography scans, and/or the like).

As further shown in FIG. 1, the transabdominal gastric drainage device 100 also includes a bolster mechanism with a cylindrical port 116 to receive the drainage tube, a disc 118 to secure the transabdominal gastric drainage device 100 on a skin side of the existing gastrostomy site of the patient, and a balloon 120 that can be inflated after insertion through the gastrostomy site to secure the transabdominal gastric drainage device 100, and subsequently deflated to allow for the transabdominal gastric drainage device 100 to be removed through the gastrostomy site. For example, as shown in FIG. 1, the hub 106 includes a plug that can be removed to allow for an inflation/deflation device to be inserted into the hub 106, which includes a tube in communication with the balloon 120. Accordingly, air may be passed into the hub 106 to inflate the balloon 120 against the stomach wall, and the cylindrical port 116 and the disc 118 may be movable along the extra-anatomical portion 108 of the drainage tube to hold the transabdominal gastric drainage device 100 in place on the skin side of the gastrostomy site. The inflation/deflation device can then be removed, and the plug replaced to seal the hub 106 and prevent the balloon 120 from deflating. Additionally, or alternatively, the inflation/deflation device may include a valve or other mechanism to prevent air from escaping through the hub 106, in which case the inflation/deflation device may remain in the hub 106 (with the valve closed) after the balloon 120 has been inflated (e.g., while the stomach contents are being drained). In either case, after the stomach contents have been sufficiently drained, the balloon 120 may be deflated using the inflation/deflation device to allow for the transabdominal gastric drainage device 100 to be removed through the gastrostomy site, and the patient's gastrostomy feeding tube can then be reinserted through the gastrostomy site. For example, to deflate the balloon 120, the inflation/deflation device may be inserted into the hub 106 and operated to draw air out of the balloon 120 through the tube connecting the hub 106 to the balloon 120 (e.g., the inflation/deflation device may be a syringe with a piston or plunger that can be pushed, depressed, and/or the like along an interior of a barrel to expel air into the balloon 120 and pulled along the interior of the barrel to withdraw air from the balloon 120).

Accordingly, in the example shown in FIG. 1, the drainage tube is specifically designed to drain gastrointestinal contents through a previously existing gastrostomy site. When a patient with a gastrostomy feeding tube in place develops obstructive symptoms, the gastrostomy feeding tube is removed, and replaced with the transabdominal gastric drainage device 100. For example, when the balloon 120 is deflated, the balloon 120 may be limp and easily passed through the gastrostomy site together with the intragastric portion 112 of the drainage tube. Furthermore, in some implementations, the drainage tube associated with the transabdominal gastric drainage device 100 may have a diameter to suit an existing size associated with the gastrostomy site (or stoma) of the patient. For example, the transabdominal gastric drainage device 100 may be manufactured in various sizes to accommodate patients that have stomas of different sizes. Furthermore, in some implementations, the drainage tube may be constructed from a flexible plastic, and the drainage tube may have more flexibility at a distal end (e.g., an end where the drainage lumen 102, the ventilation lumen 104, and the hub are open to the environment) relative to a proximal end (e.g., an end adjacent to the bolster mechanism). In this way, the proximal end may remain relatively stable and firmly held in place on the skin side of the gastrostomy site, and the additional flexibility at the distal end may allow the drainage lumen 102 to be moved relatively freely to attach to a collector bag, a suction source, and/or the like.

In some implementations, as mentioned above, the intragastric portion 112 of the drainage tube and the balloon 120 (while deflated) may be passed through the existing gastrostomy site, extending into the stomach several centimeters with the drainage holes 114 arranged throughout an intraluminal length of the intragastric portion 112 of the drainage tube to optimize drainage (e.g., the drainage holes 114 may span several inches and there may be several rows of the drainage holes 114 to remove as much fluid as possible from the stomach cavity). The balloon 120 may then be inflated through the hub 106, and a physician, nurse, medical professional, and/or the like may pull on the drainage tube with light force to secure the balloon 120 against the stomach wall. The physician, nurse, medical professional, and/or the like may then slide the bolster, which includes the cylindrical port 116 and the disc 118, along the extra-anatomical portion 108 of the drainage tube to hold the transabdominal gastric drainage device 100 in place on the skin side of the gastrostomy site. In some implementations, once the transabdominal gastric drainage device 100 has been secured in place, the stomach contents may be drained via the drainage holes 114 and the drainage lumen 102.

In particular, as shown in FIG. 1, the drainage holes 114 may be adapted to receive stomach contents, which flow into a drainage lumen 102 to allow for the stomach contents to be drained. For example, in some cases, the drainage lumen 102 may be attached to a collector bag, a waste bag, and/or the like, which may be placed below a level of the patient's stomach to allow the stomach contents to be drained using gravity. Additionally, or alternatively, the drainage lumen 102 may be attached to a suction source used to actively pull the stomach contents into the drainage holes 114 and out the drainage lumen 102. Additionally, or alternatively, a combination of suction methods can be used (e.g., gravitational drainage with intermittent active suction) to provide the benefits of active suction (e.g., fast symptom relief) while limiting potentially adverse effects from constant suction (e.g., damage to the stomach lining). Furthermore, the ventilation lumen 104 is open to air outside the body, which allows air to flow into the stomach cavity and prevent the stomach from collapsing as the stomach contents are removed through the drainage holes 114 and the drainage lumen 102. In this way, the ventilation lumen 104 may prevent potentially adverse consequences from suction on the stomach lining.

The number and arrangement of elements shown in FIG. 1 are provided merely as one or more examples. Other examples may differ from what is described with regard to FIG. 1. For example, in practice, the transabdominal gastric drainage device 100 may include additional elements, fewer elements, different elements, or differently arranged elements than those shown in FIG. 1. Additionally, or alternatively, a set of elements (e.g., one or more elements) of the transabdominal gastric drainage device 100 may perform one or more functions described as being performed by another set of elements of the transabdominal gastric drainage device 100.

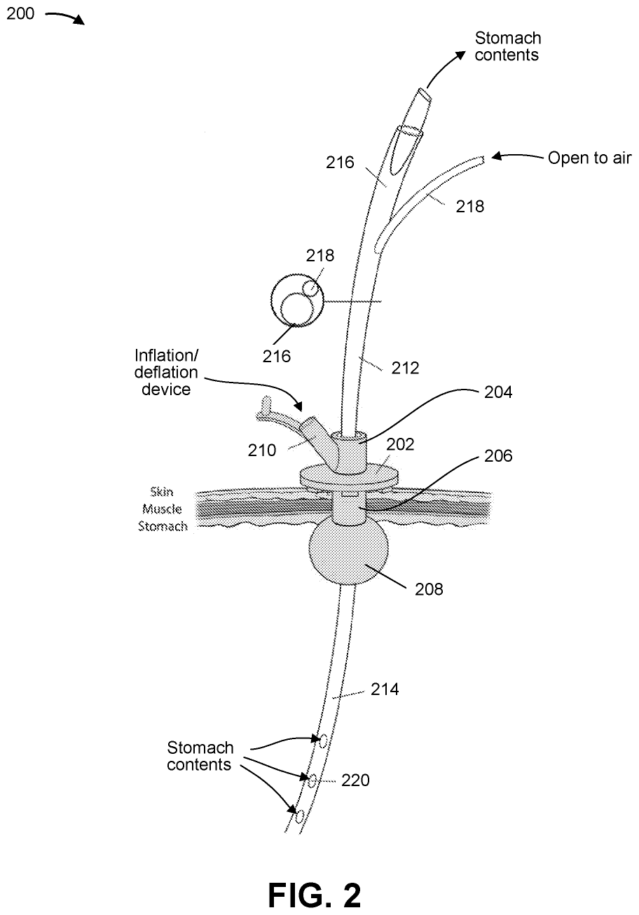

FIG. 2 is a diagram of an example implementation of a transabdominal gastric drainage system 200 described herein. As shown in FIG. 2, the transabdominal gastric drainage system 200 includes a facilitator device that includes a bolster disc 202, a cylindrical port 204 that has a flexible transabdominal portion 206, a balloon 208, and an inflation/deflation hub 210 that connects to the balloon 208 via an inflation/deflation tube. Furthermore, as shown, the inflation/deflation hub 210 includes a plug that can be removed to allow for an inflation/deflation device to be inserted into the inflation/deflation hub 210 (e.g., to inflate the balloon 208 after insertion through the gastrostomy site and/or deflate the balloon 208 prior to removal through the gastrostomy site). Furthermore, the plug can be replaced to seal the inflation/deflation hub 210 and prevent air from escaping the balloon 208 while the transabdominal gastric drainage system 200 is in place.

As further shown in FIG. 2, the transabdominal gastric drainage system 200 includes a gastric drainage tube with an extra-anatomical portion 212 and an intragastric portion 214. The gastric drainage tube includes a drainage lumen 216 that extends through the extra-anatomical portion 212 and the intragastric portion 214, passing through the cylindrical port 204 of the facilitator device and into the stomach cavity. Furthermore, the gastric drainage tube includes a ventilation lumen 218 that is open to air to permit air to pass into the stomach cavity of the patient as the stomach contents are removed via the drainage lumen 216, and the intragastric portion 112 of the gastric drainage tube has a set of drainage holes 220 that are adapted to receive the stomach contents to be drained. In some implementations, the gastric drainage tube may include a radiopaque line to make the gastric drainage tube visible in medical images. Additionally, or alternatively, the gastric drainage tube may be a standard nasogastric tube.

As further shown in FIG. 2, the cylindrical port 204 of the facilitator device may receive the gastric drainage tube, and the bolster disc 202 may operate to secure the transabdominal gastric drainage system 200 (including the gastric drainage tube and the facilitator device) on a skin side of the existing gastrostomy site of the patient. As mentioned above, the balloon 208 can be inflated after insertion through the gastrostomy site to secure the transabdominal gastric drainage system 200, and subsequently deflated to allow for the transabdominal gastric drainage system 200 to be removed through the gastrostomy site. For example, while the balloon 208 is deflated, the balloon 208 and the flexible transabdominal portion 206 of the cylindrical port may be passed through the gastrostomy site. The gastric drainage tube may then be passed through the cylindrical port 204 and into the stomach cavity. When the gastric drainage tube has been extended to the appropriate depth within the stomach cavity, the balloon 208 may be inflated and the flexible transabdominal portion 206 may tighten around the gastric drainage tube, securing the transabdominal gastric drainage system 200 in place at the gastrostomy site. In this way, the flexible transabdominal portion 206 of the cylindrical port 204 may accommodate different sizes and/or lengths for the gastric drainage tube (e.g., sizes ranging from 4 French to 18 French (French size is three times the diameter in millimeters), and with sizes and/or lengths varying for patients in different age groups). Furthermore, in a similar manner as described above with reference to FIG. 1, the bolster disc 202 may be movable along the extra-anatomical portion 212 of the gastric drainage tube to hold the transabdominal gastric drainage system 200 in place on the skin side of the gastrostomy site. In this way, the mobility of the bolster disc 202 may accommodate variations in abdominal wall thickness for different patients.

In some implementations, after the balloon 208 has been inflated to secure the transabdominal gastric drainage system 200 in place at the gastrostomy site, the inflation/deflation device can be removed from the inflation/deflation hub 210, and the plug can be replaced to prevent the balloon 208 from deflating while stomach contents are drained. Additionally, or alternatively, the inflation/deflation device may include a valve or other mechanism to prevent air from escaping through the inflation/deflation hub 210, in which case the inflation/deflation device may remain in the inflation/deflation hub 210 (with the valve closed) after the balloon 208 has been inflated (e.g., while the stomach contents are being drained). In either case, after the stomach contents have been sufficiently drained, the balloon 208 may be deflated using the inflation/deflation device to allow for the gastric drainage tube to be removed through the gastrostomy site (e.g., by decreasing pressure holding the gastric drainage tube in place). For example, to deflate the balloon 208, the inflation/deflation device may be inserted into the inflation/deflation hub 210 and operated to draw air out of the balloon 208 through the tube connecting the inflation/deflation hub 210 to the balloon 208 (e.g., the inflation/deflation device may be a syringe with a piston or plunger that can be pushed along an interior of a barrel to expel air into the balloon 208 and pulled along the interior of the barrel to withdraw air from the balloon 208). Accordingly, in some implementations, the patient's gastrostomy feeding tube can be reinserted through the gastrostomy site after the facilitator device has been removed. Additionally, or alternatively, the facilitator device may remain in place after the gastric drainage tube has been removed, and the gastric drainage tube may be replaced with a feeding tube. In this way, the facilitator device may accommodate different tubes (e.g., for feeding and drainage), which can be changed as needed without having to remove and reinsert the facilitator device.

Accordingly, in the example shown in FIG. 2, the facilitator device may allow a standard nasogastric tube to be used as the gastric drainage tube to drain gastrointestinal contents through a previously existing gastrostomy site. When a patient with a gastrostomy feeding tube in place develops obstructive symptoms, the gastrostomy feeding tube is removed, and the (deflated) balloon 208 and the flexible transabdominal portion 206 of the facilitator device are passed through the gastrostomy site. For example, when the balloon 208 is deflated, the balloon 208 may be limp and the flexible transabdominal portion 206 may be in a collapsed state that allows the balloon 208 and the flexible transabdominal portion 206 to easily pass through the gastrostomy site. The gastric drainage tube can then be passed through the cylindrical port 204 and into the stomach cavity, extending into the stomach several centimeters with the drainage holes 220 arranged throughout an intraluminal length of the intragastric portion 214 of the gastric drainage tube to optimize drainage. The balloon 208 may then be inflated through the inflation/deflation hub 210, causing the flexible transabdominal portion 206 of the facilitator device to tighten around the gastric drainage tube. The gastric drainage tube may then be pulled with light force to secure the balloon 208 against the stomach wall, and the bolster disc 202 can be moved along the extra-anatomical portion 212 of the gastric drainage tube to hold the transabdominal gastric drainage system 200 in place on the skin side of the gastrostomy site. In some implementations, once the transabdominal gastric drainage system 200 has been secured in place, the stomach contents may be drained via the drainage holes 220 and the drainage lumen 216 (e.g., using gravity, an active suction source, and/or the like).

In particular, as shown in FIG. 2, the drainage holes 220 may be adapted to receive stomach contents, which flow into the drainage lumen 216 to allow for the stomach contents to be drained. For example, in some cases, the drainage lumen 216 may be attached to a collector bag, a waste bag, and/or the like, which may be placed below a level of the patient's stomach to allow the stomach contents to be drained using gravity. Additionally, or alternatively, the drainage lumen 216 may be attached to a suction source configured to actively pull the stomach contents into the drainage holes and out the drainage lumen 216. Additionally, or alternatively, a combination of suction methods can be used (e.g., gravitational drainage with intermittent active suction) to provide the benefits of active suction (e.g., fast symptom relief) while limiting potentially adverse effects from constant suction (e.g., damage to the stomach lining). Furthermore, the ventilation lumen 218 is open to air outside the body, which allows air to flow into the stomach cavity and prevent the stomach from collapsing as the stomach contents are removed through the drainage holes 220 and the drainage lumen 216. In this way, the ventilation lumen 218 may prevent potentially adverse consequences from suction on the stomach lining. After the stomach contents have been suitably drained, the balloon 208 can be deflated, which causes the flexible transabdominal portion 206 of the facilitator device to collapse, permitting the gastric drainage tube and the facilitator device to be removed through the gastrostomy site. The patient's gastrostomy feeding tube can then be reinserted as necessary, and subsequently removed and replaced with the transabdominal gastric drainage system 200 to further treat obstructive systems as the need arises.

The number and arrangement of elements shown in FIG. 2 are provided merely as one or more examples. Other examples may differ from what is described with regard to FIG. 2. For example, in practice, the transabdominal gastric drainage system 200 may include additional elements and/or devices, fewer elements and/or devices, different elements and/or devices, or differently arranged elements and/or devices than those shown in FIG. 2. Additionally, or alternatively, a set of elements and/or devices (e.g., one or more elements and/or devices) of the transabdominal gastric drainage system 200 may perform one or more functions described as being performed by another set of elements and/or devices of the transabdominal gastric drainage system 200.

The foregoing disclosure provides illustration and description, but is not intended to be exhaustive or to limit the implementations to the precise forms disclosed. Modifications and variations may be made in light of the above disclosure or may be acquired from practice of the implementations.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A device, comprising:
a tube including a drainage lumen and a hub adapted to receive an inflation device,
wherein the tube includes an intragastric portion having a set of holes to permit gastrointestinal contents to pass from a stomach cavity into the drainage lumen;
a bolster including a cylindrical port adapted to receive the tube and a disc adapted to hold the tube in place on a skin side of a gastrostomy site; and
a balloon formed around the intragastric portion of the tube at a location proximal to a transabdominal portion of the tube,
wherein the balloon and the intragastric portion of the tube are adapted to pass through the gastrostomy site into the stomach cavity while the balloon is deflated, and
wherein the balloon is inflatable within the stomach cavity using the inflation device to secure the transabdominal portion of the tube within the gastrostomy site.

2. The device of claim 1, wherein the tube includes a ventilation lumen open to air to permit air to pass into the stomach cavity as the gastrointestinal contents pass into the drainage lumen.

3. The device of claim 1, wherein the tube includes an extra-anatomical portion having a first end that is proximal to the gastrostomy site and a second end that is distal to the gastrostomy site and more flexible than the first end.

4. The device of claim 1, wherein the balloon is deflatable within the stomach cavity using the inflation device to permit the balloon and the intragastric portion of the tube to be removed from the stomach cavity through the gastrostomy site.

5. The device of claim 1, wherein the set of holes includes multiple holes that are arranged throughout an intraluminal length of the intragastric portion of the tube.

6. The device of claim 1, wherein the drainage lumen is connected to a suction source or left open to gravity to permit the gastrointestinal contents to be drained from the stomach cavity.

7. The device of claim 1, wherein the tube and the cylindrical port of the bolster have a width based on a size of the gastrostomy site.

8. The device of claim 1, wherein the tube further includes an inflation tube coupling the balloon to the hub adapted to receive the inflation device.

9. The device of claim 1, wherein the hub includes a plug that can be removed to receive the inflation device and replaced to seal the hub after the balloon has been inflated and the inflation device has been removed.

10. The device of claim 1, wherein the tube further includes a radiopaque line.

11. A device, comprising:
a cylindrical port adapted to receive a nasogastric tube,
wherein the cylindrical port includes an extra-anatomical portion coupled to a hub adapted to receive an inflation device, and
wherein the cylindrical port includes a flexible transabdominal portion;
a bolster disc adapted to hold the nasogastric tube in place on a skin side of a gastrostomy site; and
a balloon in communication with the hub,
wherein the balloon and the flexible transabdominal portion of the cylindrical port are adapted to pass through the gastrostomy site while the balloon is deflated, and
wherein the balloon is inflatable within a stomach cavity using the inflation device to secure the device and the nasogastric tube within the gastrostomy site.

12. The device of claim 11, wherein the flexible transabdominal portion is collapsible to accommodate different sizes for the nasogastric tube.

13. The device of claim 11, wherein the balloon is deflatable within the stomach cavity using the inflation device to permit the device to be removed from the gastrostomy site.

14. The device of claim 11, further comprising an inflation tube coupling the balloon to the hub adapted to receive the inflation device.

15. The device of claim 11, wherein the hub includes a plug that can be removed to receive the inflation device and replaced to seal the hub after the balloon has been inflated.

16. A system, comprising:
a gastric drainage tube; and
a facilitator device including:
a cylindrical port adapted to receive the gastric drainage tube,
wherein the cylindrical port includes an extra-anatomical portion coupled to a hub adapted to receive an inflation device, and
wherein the cylindrical port includes a flexible transabdominal portion;
a bolster disc adapted to hold the gastric drainage tube in place on a skin side of a gastrostomy site; and
a balloon in communication with the hub,
wherein the balloon and the flexible transabdominal portion of the cylindrical port are adapted to pass through the gastrostomy site while the balloon is deflated, and
wherein the balloon is inflatable within a stomach cavity using the inflation device after the balloon has passed through the gastrostomy site to secure the facilitator device and the gastric drainage tube within the gastrostomy site.

17. The system of claim 16, wherein the gastric drainage tube includes a drainage lumen and an intragastric portion having a set of holes to permit gastrointestinal contents to pass from the stomach cavity into the drainage lumen.

18. The system of claim 17, wherein the set of holes includes multiple holes that are arranged throughout an intraluminal length of the intragastric portion of the gastric drainage tube.

19. The system of claim 16, wherein the gastric drainage tube includes a radiopaque line.

20. The system of claim 16, wherein the gastric drainage tube includes an extra-anatomical portion having a first end that is proximal to the gastrostomy site and a second end that is distal to the gastrostomy site and more flexible than the first end.
